(19) United States
(12) Patent Application Publication
(10) Pub. No.: US 2026/0355103 A1
(43) Pub. Date: Nov. 12, 2026

(54) NATURAL SUBSTANCE-BASED HAIR DYEING METHOD

(71) Applicant: HENKEL AG & CO. KGAA, Düsseldorf (DE)

(72) Inventors: Skrollan KLAAS, Hamburg (DE); Nele DALLMANN, Hamburg (DE); Jana Marie ROESEKE, Tornesch (DE); Fabian STRASKE, Hamburg (DE); Thomas HIPPE, Appen (DE)

(21) Appl. No.: 19/120,828

(22) PCT Filed: Sep. 22, 2023
(86) PCT No.: PCT/EP2023/076185

§ 371 Date: Apr. 14, 2025

(30) Foreign Application Priority Data
Oct. 17, 2022 (DE) 10 2022 210 944.9

(51) Int. Cl. A61K 8/9789; A61K 8/23; A61K 8/365; A61Q 5/10

(52) U.S. Cl. CPC A61K 8/9789; A61K 8/23; A61K 8/365; A61Q 5/10; A61K 2800/42; A61K 2800/81; A61K 2800/87; A61K 2800/88

(57) ABSTRACT

A method for dyeing keratin-containing fibers, in particular human hair, using Camellia sinensis leaf powder and one or more silver salts, and to a kit for carrying out the dyeing method, are described

The invention relates to a method for dyeing keratin-containing fibers, in particular human hair, using a leaf powder of Camellia sinensis and one or more silver salts, and to a kit for carrying out this dyeing method.

The desire to change one's hair color is a great need for many consumers. To satisfy this need, the cosmetic industry provides a diverse range of products. Hair dyes that achieve particularly long-lasting coloring with high coverage are usually oxidation dyes. These use oxidizing agents, which can damage the hair structure. Certain cationic direct azo dyes are also capable of producing hair color changes with excellent fastness properties. However, said azo dyes are synthetic dyes.

There is a demand among a growing number of consumers for hair dyes and hair dyeing methods based on natural dyes, even though these dyes and methods are often inferior to the aforementioned dyes and methods in terms of fastness properties, coverage, and color variety.

### PRIOR ART

In addition to dyeing with henna, which comes from the Lawsonia inermis plant, it is also already known to dye hair with parts of the Camellia sinensis plant, especially with the leaves of Camellia sinensis; see KR20090056479A.

The use of salts of certain transition metals, in particular such as the salts of silver, for natural hair dyeing is also known in the prior art; see JP2013001673A, KR20150027578A or EP327345A2.

In the dyeing methods known in the prior art that use metal salts or parts of the Camellia sinensis plant, only a black color is usually achieved.

EP3192488A1 discloses an oxidative hair dyeing method that uses an aqueous tea powder solution for color-fixing after-treatment. WO2021110768A1 discloses matcha as a component of a water-free dry shampoo.

### Object

The object of the present invention was to provide a method for dyeing keratin-containing fibers, in particular human hair, using naturally occurring substances, which can be used to create a wider range of dye colors.

Surprisingly, it was found that with the dyeing methods and kits described in the claims, using a leaf powder of Camellia sinensis and one or more silver salts, dyes with a wider variety of colors can be achieved.

The present invention therefore firstly relates to a method for non-oxidatively dyeing keratin fibers, in particular human hair, which comprises the following method steps in the order given:
i. Providing a composition C containing powdered leaves of Camellia sinensis in a cosmetic carrier,
ii. Applying composition C to the keratin fibers to be dyed,
iii. Allowing composition C to act for a period of 1 to 60 minutes,
iv. Rinsing off composition C,
v. Optionally drying the keratin fibers,
vi. Subsequently treating the keratin fibers with a composition S containing an aqueous solution of a silver salt for a period of 0.5 to 60 minutes,
vii. Rinsing off composition S which contains silver salt, and
viii. Optionally drying the keratin fibers,
   wherein no oxidizing agents other than atmospheric oxygen are used in the method.
   Effect of Electromagnetic Radiation on the Hair Treated with Camellia sinensis and Silver Salt

Preferred dyeing methods according to the invention are characterized in that the keratin fibers are exposed to electromagnetic radiation following method step vii, or method step viii. This radiation exposure preferably lasts for 0.5 to 60 minutes, particularly preferably 1 to 45 minutes, extremely preferably 5 to 30 minutes, more extremely preferably 10 to 20 minutes.

Preferred electromagnetic radiation according to the invention, to which the keratin fibers treated with Camellia sinensis and silver salt are exposed in optional step (ix) of the method according to the invention, is UV/VIS radiation, particularly preferably UV/VIS radiation in the wavelength range from 200 to 800 nm, extremely preferably in the wavelength range from 290 to 700 nm.

Preferred dyeing methods according to the invention are characterized in that the keratin fibers, following method step vii, or method step viii., are exposed to electromagnetic radiation, which is preferably selected from UV/VIS radiation in the wavelength range from 200 to 800 nm, in a method step (ix) for a period of 0.5 to 60 minutes, preferably 1 to 45 minutes, particularly preferably 5 to 30 minutes, extremely preferably 10 to 20 minutes.

As electromagnetic radiation in the wavelength range of from 200 nm to 800 nm, UV/VIS radiation includes the UV range (ultraviolet range) and the VIS range (visible range).

UV radiation (ultraviolet radiation) is electromagnetic waves having a wavelength of 380 to 10 nm or a frequency of approximately 790 THz to 30 PHz. The energy of a single quantum of light is in the range of approximately 3.3 eV (380 nm) to approximately 124 eV (10 nm).

The light visible to the human eye is the part of the electromagnetic spectrum having wavelengths between approximately 380 and 780 nm. Light having a wavelength of 800 nm is still just about visible.

Within the meaning of the invention, exposure to electromagnetic radiation means, on the one hand, exposure to radiation from a natural radiation source and, on the other hand, irradiation with an artificial light source which emits the electromagnetic radiation in the desired wavelength range.

Exposing the keratin fibers treated with Camellia sinensis and silver salt to daylight may also be understood as exposure to electromagnetic radiation or irradiation within the meaning of the invention.

In the dyeing method preferred according to the invention, the keratin fibers to be treated are first treated with Camellia sinensis and then with a silver salt and rinsed with water one after the other. It is preferable that the keratin fibers are dried after the final rinsing step, preferably first with a towel, then if necessary with a hairdryer or a drying hood, or left to air dry. Particularly preferred dyeing methods according to the invention are characterized in that the keratin fibers, after completion of method step viii., have a water content of 1-42 wt. %, preferably 3-30 wt. %, particularly preferably 5-25 wt. %, extremely preferably 8-15 wt. % of water in each case based on their weight. After the optional drying step, the keratin fibers are exposed to electromagnetic radiation.

The source of this electromagnetic radiation can be artificial or natural. Artificial or natural electromagnetic radiation can be continuous or discontinuous.

The electromagnetic radiation used is preferably light radiation in the wavelength range from 200 nm to 800 nm. Within the context of the present invention, the term “artificial light radiation” means light radiation that is different from natural daylight, i.e. light produced by the sun. In other words, natural daylight, i.e. daylight produced by the sun, is not artificial light radiation.

The term “natural light radiation” means radiation whose only source of light is the daylight produced by the sun.

Preferably, the hair is irradiated with natural light radiation having a wavelength in the range of 360 to 600 nm, preferably in the range of 375 to 550 nm, particularly preferably in the range of 400 to 480 nm.

It is also preferred to irradiate the hair with artificial light radiation having a wavelength in the range from 360 to 600 nm, preferably in the range from 375 to 550 nm, particularly preferably in the range from 400 to 480 nm.

Preferably, the natural light radiation used has an energy quantity per unit area of up to or equal to 1 J/cm², more preferably greater than 1 J/cm², even more preferably in the range of 1.001 to 100 J/cm², even more preferably in the range of 2 to 50 J/cm², particularly preferably in the range of 3 to 10 J/cm².

Preferably, the artificial light radiation used has an energy quantity per unit area of up to or equal to 1 J/cm², more preferably greater than 1 J/cm², even more preferably in the range of 1.001 to 100 J/cm², even more preferably in the range of 2 to 50 J/cm², particularly preferably in the range of 3 to 10 J/cm₂.

Preferably, the natural light radiation used has a luminous efficacy of 50 to 100 lm/W (lumens per watt), preferably 70 to 85 lm/W.

Preferably, the artificial light radiation used has a luminous efficacy of 50 to 100 lm/W (lumens per watt), preferably 70 to 85 lm/W.

Preferably, the natural light radiation used has a luminous flux of 50 to 100 lm/W (lumens per watt), preferably 70 to 85 lm/W.

Preferably, the artificial light radiation used has a luminous flux of 3500 to 8000 lumens, preferably 4000 to 6000 lumens.

The artificial light radiation is preferably generated using a device selected from arc lamps such as xenon lamps and mercury lamps, fluorescent lamps, incandescent lamps such as halogen lamps, light-emitting diodes (LEDs), organic light-emitting diodes (OLEDs) and lasers.

Examples include Golite BLU products from Philips, the Energylight HF 3319/01 lamp from Philips, the Dayvia White and Messa lamps from Solvital, the Lumino Plus lamp from Laanaform, the Medibeam lamp from Medibeam, the M-LED 01 lamp from Meimed, the Lifemax Light Pod lamp from Lifemax, the Lite-Pad lamp from Reicorp, the Omnilux Clear-U and New-U lamps from Omnilux, the 1000 W xenon arc lamp from Lot-Oriel, and the Camag Box 3 (4×8 W) lamp from Camag.

For the application according to the invention to the keratin fibers, the plant leaves of Camellia sinensis are dried and ground and used as a leaf powder. Leaf powders that are preferred according to the invention have a particle size of less than 500 μm, particularly preferably in the range of 120 μm-200 μm, extremely preferably in the range of 150 μm-180 μm. Other leaf powders preferred according to the invention have a bulk density in the range of 0.20-0.60 g/cm³, particularly preferably in the range of 0.25-0.40 g/cm³. Further leaf powders that are preferred according to the invention have, based on their weight, a moisture content of less than 10 wt. %, preferably 0.1-6.0 wt. %, particularly preferably 0.2-3.0 wt. %. The moisture content is determined after 3 hours of drying at 105 °C.

Further compositions C that are preferred according to the invention for the treatment of keratin fibers are characterized in that the leaf powder of Camellia sinensis has a particle size of less than 500 μm, particularly preferably in the range of 120 μm-200 μm, extremely preferably in the range of 150 μm-180 μm, and a bulk density in the range of 0.20-0.60 g/cm³, particularly preferably in the range of 0.25-0.40 g/cm³, and has, based on its weight, a moisture content of less than 10 wt. %, preferably 0.1-6.0 wt. %, particularly preferably 0.2-3.0 wt. %.

The Camellia sinensis leaves contained in a composition C that is preferably used according to the invention can preferably be matcha leaves. Matcha is understood to mean green tea leaves ground into powder, obtained from the plant Camellia sinensis. Green tea leaves used to make matcha are harvested from tea shrubs that are shaded from approximately four weeks before harvest. For this purpose, the tea shrubs are covered with opaque nets, which extends the ripening time. This creates a dark-green leaf. After harvesting, the green tea leaves are steamed and dried. After removing all coarse leaf vessels, the fine leaf tissue or leaf flesh is ground into a fine powder, called matcha. Matcha preferably used according to the invention is water-soluble.

Dyeing methods that are preferred according to the invention are characterized in that the leaf powder of Camellia sinensis is contained in an amount of 0.2-50 wt. %, preferably 0.5-20 wt. %, particularly preferably 0.8-10 wt. %, extremely preferably 1-2 wt. %, based on the weight of composition C. Dyeing methods that are particularly preferred according to the invention are characterized in that matcha is contained in an amount of 0.2-50 wt. %, preferably 0.5-20 wt. %, particularly preferably 0.8-10 wt. %, extremely preferably 1-2 wt. %, based on the weight of composition C.

Further dyeing methods that are preferred according to the invention are characterized in that composition C containing Camellia sinensis has a pH of 2.0 to 8.0, preferably 2.5 to 7.5, particularly preferably 3.0 to 7.0, measured in each case at 20 °C.

Preferred dyeing methods according to the invention are characterized in that the at least one silver salt with which the keratin fibers are treated after treatment with Camellia sinensis is selected from silver nitrate, silver sulfate, silver citrate, silver dihydrogen citrate, silver lactate, silver acetate, silver malate, silver succinate, silver tartrate, silver mandelate, silver salicylate, silver gluconate, silver adipate and silver galactarate, as well as mixtures of these salts. Silver salts which are extremely preferred according to the invention are selected from silver nitrate, silver sulfate, silver citrate, silver dihydrogen citrate and silver lactate, as well as mixtures of these salts.

Dyeing methods that are particularly preferred according to the invention are characterized in that the at least one silver salt is contained in a total amount of 0.05-2 wt. %, preferably 0.1-1.5 wt. %, particularly preferably 0.2-0.8 wt. %, extremely preferably 0.3-0.5 wt. %, in each case based on the weight of composition S.

Dyeing methods which are extremely preferred according to the invention are characterized in that the at least one silver salt is contained in a total amount of 0.05-2 wt. %, preferably 0.1-1.5 wt. %, particularly preferably 0.2-0.8 wt. %, extremely preferably 0.3-0.5 wt. %, in each case based on the weight of composition S, wherein the silver salt is selected from silver nitrate, silver sulfate, silver citrate, silver dihydrogen citrate, silver lactate, silver acetate, silver malate, silver succinate, silver tartrate, silver mandelate, silver salicylate, silver gluconate, silver adipate and silver galactarate, as well as mixtures of these salts.

Further dyeing methods which are extremely preferred according to the invention are characterized in that the at least one silver salt is contained in a total amount of 0.05-2 wt. %, preferably 0.1-1.5 wt. %, particularly preferably 0.2-0.8 wt. %, extremely preferably 0.3-0.5 wt. %, in each case based on the weight of composition S, wherein the silver salt is selected from silver nitrate, silver sulfate, silver citrate, silver dihydrogen citrate and silver lactate, as well as mixtures of these salts.

Further dyeing processes that are preferred according to the invention are characterized in that the silver salt-containing composition S has a pH of 2.0 to 8.0, preferably 3.0 to 7.5, particularly preferably 4.0 to 7.0, extremely preferably 4.5 to 5.0, in each case measured at 20 °C.

In a preferred embodiment of the dyeing method according to the invention, the desired pH of the aqueous compositions (C) or (S) is adjusted independently of one another by means of an acid or a base. Preferred acids are selected from citric acid, lactic acid, gluconic acid, acetic acid, propionic acid, oxalic acid, malonic acid, succinic acid, glutaric acid, galactaric acid (mucic acid), tartaric acid, malic acid, sulfuric acid and phosphoric acid as well as mixtures of these acids. Preferred bases are sodium hydroxide, potassium hydroxide, arginine, lysine, monoethanolamine, triethanolamine, 2-amino-2-methylpropan-1-ol and mixtures of these bases.

In a further preferred embodiment of the dyeing method according to the invention, the desired pH of the aqueous compositions (C) or (S) is adjusted independently of one another with the aid of a buffer system selected from a mixture of a medium-strength or weak acid with its conjugated or corresponding base (or the corresponding salt) and a mixture of a medium-strength or weak base with its conjugated or corresponding acid.

Corresponding acid-base pairs that are preferably suitable according to the invention are those which stabilize the aqueous compositions (C) or (S) used according to the invention independently of one another in the pH range from 2.0 to 8.0, preferably from 2.5 to 7.5, particularly preferably from 3.0 to 7.0, in each case measured at 20 °C.

Particularly preferred buffer systems according to the invention for the aqueous composition (C) used according to the invention are selected from
Ammonia/ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate, ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride, Mixtures of hydrogen phosphate and dihydrogen phosphate, in particular the alkali metal salts of hydrogen phosphate and dihydrogen phosphate, particularly preferably the sodium salts and/or the potassium salts of hydrogen phosphate and dihydrogen phosphate,
Mixtures of alkali metal hydrogen carbonate with alkali metal carbonate, in particular mixtures of sodium hydrogen carbonate or potassium hydrogen carbonate with sodium carbonate or potassium carbonate,
Mixtures of citric acid and its salts, in particular alkali metal citrates, in particular sodium salts, in particular trisodium citrate,
Mixtures of tartaric acid and its salts, in particular alkali metal tartrates, in particular potassium salts, in particular potassium hydrogen tartrate,
Mixtures of phthalic acid and its salts, in particular potassium salts, in particular potassium hydrogen phthalate,
Mixtures of lactic acid and its salts, in particular lactic acid/sodium lactate mixtures,
Mixtures of gluconic acid and its salts, in particular gluconic acid/sodium gluconate mixtures,
Mixtures of succinic acid and its salts, in particular sodium salts, in particular sodium hydrogen succinate and disodium succinate, and
Mixtures of malic acid and its salts, in particular sodium salts, in particular sodium hydrogen malate and disodium malate, as well as
Ammonia/ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate, ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride.

Other buffer systems, e.g. acetic acid/sodium acetate, are in principle also suitable according to the invention. However, due to the vinegary smell, such a buffer is not acceptable for the preparation of a cosmetic market product.

To vary the pH, further dyes and dyeing methods that are preferred according to the invention are therefore characterized in that the aqueous compositions (C) or (S) contain, independently of one another, a buffer system for pH adjustment, selected from a mixture of a medium-strength or weak acid or base with its conjugated or corresponding base or corresponding acid.

Further dyeing methods that are preferred according to the invention are characterized in that the aqueous compositions (C) or (S) contain, independently of one another, a buffer system selected from an ammonia/ammonium salt mixture for adjusting the pH in the basic range. Preferred ammonium salts which buffer the strongly basic pH of the aqueous ammonia solution to a less basic pH are selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate, ammonium lactate, as well as mixtures of these ammonium salts. Ammonium chloride, ammonium hydrogen sulfate, ammonium sulfate, ammonium dihydrogen phosphate, diammonium hydrogen phosphate, ammonium phosphate, ammonium bicarbonate and ammonium carbonate are particularly preferred. Ammonium chloride is extremely preferred.

However, in principle, other buffer systems are also suitable.

Further dyeing methods that are preferred according to the invention are characterized in that the aqueous compositions (C) or (S) contain, independently of one another, a buffer system selected from a hydrogen phosphate salt/dihydrogen phosphate salt mixture for adjusting the pH. Suitable salts are the sodium salts and the potassium salts of hydrogen phosphate and dihydrogen phosphate. With hydrogen phosphate salt/dihydrogen phosphate salt mixtures, pH values in the range of 7.1 to approximately 8.2 can be set.

For higher pH values, mixtures of sodium hydrogen carbonate or potassium hydrogen carbonate with sodium carbonate or potassium carbonate are suitable. Further dyeing methods that are preferred according to the invention are therefore characterized in that the aqueous compositions (C) or (S) contain, independently of one another, a buffer system selected from a mixture of sodium hydrogen carbonate or potassium hydrogen carbonate with sodium carbonate or potassium carbonate for adjusting the pH. Suitable salts are the sodium salts and the potassium salts of hydrogen carbonate and carbonate. With hydrogen carbonate salt/carbonate salt mixtures, pH values in the range of approximately 9.0 to approximately 11.0 can be set.

Those skilled in the art can find out the appropriate weights of buffer salts for setting the desired pH from relevant manuals.

Further dyeing methods that are preferred according to the invention are therefore characterized in that the aqueous compositions (C) or (S) contain, independently of one another, an acid or a base for adjusting the pH, without the corresponding base or acid being contained each time. The aforementioned acids and bases are also suitable for this purpose according to the invention.

The compositions C and S used according to the invention contain the corresponding obligatory component, i.e. the powdered leaves of Camellia sinensis or the at least one silver salt, in a cosmetic carrier. In a first embodiment preferred according to the invention, the carrier is water.

Further dyeing methods that are preferred according to the invention are characterized in that composition C containing Camellia sinensis contains water in an amount of 30.0-99.8 wt. %, preferably 50.0-98.0 wt. %, particularly preferably 70.0-90.0 wt. %, extremely preferably 80.0-86.0 wt. %, based on the weight of composition C.

Further dyeing methods that are preferred according to the invention are characterized in that composition S containing silver salt contains water in an amount of 30.0-99.8 wt. %, preferably 50.0-98.0 wt. %, particularly preferably 70.0-90.0 wt. %, extremely preferably 80.0-86.0 wt. %, based on the weight of composition S.

In order to optimize the applicability of compositions C and S to the keratin fibers and their application time, it is preferred for compositions C and S to have a thicker consistency. Preferably used compositions C and S are, each independently of one another, preferably in the form of a gel, cream or paste. Such carriers ensure a homogeneous distribution and a sufficient application time of compositions C and S on the keratin fibers.

The compositions (C) and (S) used according to the invention and preferred according to the invention can optionally contain further additives in order to optimize the application properties of these compositions. Preferred additives are in particular thickeners, which ensure that compositions (C) and (S) remain on the hair better during application.

Compositions (C) and (S) that are particularly preferably used according to the invention each contain, independently of one another, at least one or more hydrophilic thickeners, which is or are preferably selected from polysaccharides that can be chemically and/or physically modified. Compounds from the group of polysaccharides are particularly preferred according to the invention as hydrophilic thickeners, since the basic structures of the polysaccharides are of natural origin and biodegradable. Preferred hydrophilic polysaccharide thickeners are selected from celluloses, cellulose ethers of C₁-C₄ alcohols, cellulose esters, xanthan gum, alginic acids (and their corresponding physiologically acceptable salts, the alginates), agar-agar (with the polysaccharide agarose that is present as the main component of agar-agar), starch fractions and starch derivatives such as amylose, amylopectin and dextrins, gum karaya, locust bean gum, gum arabic, pectins, dextrans and guar gum, and mixtures thereof.

Cellulose ethers of C₁-C₄ alcohols and cellulose esters that are preferred according to the invention are selected from methyl celluloses, ethyl celluloses, hydroxyalkyl celluloses (such as hydroxyethyl cellulose), methylhydroxyalkyl celluloses, and carboxymethyl celluloses (such as those with the INCI name cellulose gum) and also their physiologically acceptable salts.

In preferred embodiments, the hydrophilic thickener present is xanthan gum, with a view to reliable viscosity adjustment and residue-free application to keratin fibers and the scalp. With regard to reliably adjusting the viscosity and for residue-free application to keratin fibers and the scalp, in further preferred embodiments carboxymethyl cellulose (preferably sodium carboxymethyl cellulose with the INCI name cellulose gum) is contained as a hydrophilic thickener. Carboxymethyl cellulose can be contained in a preferred embodiment as the sole hydrophilic thickener. Particular preference is given to a combination of sodium carboxymethyl cellulose and hydroxyethyl cellulose. The combination of sodium carboxymethyl cellulose and xanthan may also be preferred according to the invention.

Compositions (C) and (S) that are particularly preferably used according to the invention each contain, independently of one another, at least one hydrophilic thickener in a total amount of 0.1 to 5 wt. %, preferably 0.5 to 4 wt. %, more preferably 1 to 3.5 wt. %, and very particularly preferably 1.2 to 2 wt. %, in each case based on the weight of the particular composition (C) or composition (S).

Compositions (C) and (S) that are particularly preferably used according to the invention each contain, independently of one another, at least one organic solvent that has a phenyl group in the molecule. Preferably, this solvent is selected from phenoxyethanol, benzyl alcohol, and mixtures thereof. Surprisingly, it was found that such aromatic solvents can have a positive effect on the dyeing results of the dyeing method according to the invention; this was observed in particular when composition (C) contains such an aromatic solvent.

In a further preferred embodiment of the present invention, compositions (C) that are preferred according to the invention contain, in each case based on their weight, 0.1 to 3 wt. %, preferably 0.5 to 2.5 wt. %, more preferably 0.8 to 1.0 wt. %, of at least one organic solvent that has a phenyl group in the molecule. In a further preferred embodiment of the present invention, compositions (C) according to the invention contain, in each case based on their weight, 0.1 to 3 wt. %, preferably 0.5 to 2.5 wt. %, more preferably 0.8 to 1.0 wt. %, of at least one organic solvent selected from phenoxyethanol, benzyl alcohol and mixtures thereof.

Further compositions (C) and (S) that are particularly preferably used according to the invention each contain, independently of one another, at least one aliphatic solvent selected from C₁-C₄ alkanols and C₂-C₁₂ polyols, in particular selected from ethanol, isopropanol, n-propanol, ethylene glycol, 1,2-propanediol, glycerin, diethylene glycol, dipropylene glycol, 1,3-butylene glycol, 1,2-hexanediol, 1,6-hexanediol, and 1,2-octanediol, and mixtures of these solvents.

Further compositions (C) and (S) that are particularly preferably used according to the invention each contain, independently of one another, at least one aliphatic solvent selected from C₁-C₄ alkanols and C₂-C₁₂ polyols in a total amount of 0.01-60 wt. %, preferably 0.1-30 wt. %, particularly preferably 0.5-20 wt. %, extremely preferably 1-10 wt. %, further preferably 2-5 wt. %, in each case based on the weight of compositions (C) and (S).

Other compositions (C) and (S) that are particularly preferred according to the invention are characterized in that they, independently of one another, do not contain an aliphatic solvent selected from C₁-C₄ alkanols and C₂-C₁₂ polyols.

Compositions (C) and (S) which are particularly preferably used according to the invention each contain, independently of one another, at least one oil. Preferred cosmetic oils are selected from natural and synthetic hydrocarbons, particularly preferably from paraffin oils, C₁₈-C₃₀ isoparaffins, in particular isoeicosane, polyisobutenes and polydecenes, C₈-C₁₆ isoparaffins, and 1,3-di-(2-ethylhexyl)cyclohexane; the benzoic acid esters of linear or branched C₈₋₂₂ alkanols; fatty alcohols having 6-30 carbon atoms that are unsaturated or branched and saturated or branched and unsaturated; triglycerides of linear or branched, saturated or unsaturated, optionally hydroxylated C₈₋₃₀ fatty acids, in particular natural oils; the dicarboxylic acid esters of linear or branched C₂-C₁₀ alkanols; the esters of the linear or branched, saturated or unsaturated fatty alcohols having 2-30 carbon atoms with linear or branched, saturated or unsaturated fatty acids having 2-30 carbon atoms, which can be hydroxylated; the addition products of 1 to 5 propylene oxide units to mono- or polyvalent C₈₋₂₂ alkanols; the addition products of at least 6 ethylene oxide units and/or propylene oxide units to mono- or polyvalent C₃₋₂₂ alkanols; the C₈-C₂₂ fatty alcohol esters of monovalent or polyvalent C₂-C₇ hydroxycarboxylic acids; the symmetrical, unsymmetrical, or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched, or cyclic C₂-C₁₈ alkanols or with polyvalent linear or branched C₂-C₆ alkanols; silicone oils, and mixtures of the aforementioned substances.

Further compositions (C) and (S) which are particularly preferably used according to the invention contain, each independently of one another, at least one surfactant or one emulsifier.

Surfactants and emulsifiers in the sense of the present application are amphiphilic (bifunctional) compounds that consist of at least one hydrophobic and at least one hydrophilic molecule part. The hydrophobic group is preferably a hydrocarbon chain having 8-28 carbon atoms, which can be saturated or unsaturated, linear or branched. This C₈-C₂₈ alkyl chain is particularly preferably linear. The basic properties of the surfactants and emulsifiers are oriented absorption at boundary surfaces and also the aggregation to micelles and the formation of lyotropic phases. When selecting surfactants that are suitable according to the invention, it may be preferable to use a mixture of surfactants in order to optimally adjust the stability of each of compositions (C) and (S) used according to the invention independently of one another. Preferred surfactants and emulsifiers are selected from anionic, cationic, zwitterionic, amphoteric, and nonionic surfactants and emulsifiers and mixtures of these substances.

Other compositions (C) and (S) that are particularly preferably used according to the invention each contain, independently of one another, at least one linear saturated alkanol having 12-30 carbon atoms.

Preferred linear saturated alkanols having 12-30 carbon atoms, in particular having 16-22 carbon atoms, are selected from cetyl alcohol, stearyl alcohol, arachidyl alcohol, behenyl alcohol, and lanolin alcohol, and mixtures of these alkanols. Alkanol mixtures that are particularly preferred according to the invention are those obtained from the industrial hydrogenation of vegetable and animal fatty acids. Preferably, the total amount of at least one linear saturated alkanol having 12-30 carbon atoms is 0.1-20 wt. %, preferably 0.5-16.5 wt. %, and particularly preferably 3-10 wt. %, in each case based on the weight of composition (C) or (S).

Preferred kits and dyeing methods according to the invention are further characterized in that no hydrogen peroxide is used therein.

The statements made regarding composition (C) and composition (S), which form part of the kit according to the invention, apply, mutatis mutandis, to other preferred embodiments of the dyeing method according to the invention.

In order to preserve the hair-protecting potential of the natural dyes, the claimed method is preferably limited to those methods in which the keratin fibers have not been treated with an oxidizing agent within a period of up to 7 days prior to the application of compositions (C) and (S) used according to the invention.

The oxidizing agents which are usually used in hair cosmetics but which, according to the invention, are not intended to be used to treat hair, not even as a pre-treatment, include hydrogen peroxide, persulfates, perbromates, percarbonates, perborates and percarbamides. The oxygen contained in the ambient air does not constitute an oxidizing agent within the context of the invention.

In order to preserve the hair-protecting potential of the natural dyes, methods preferred according to the invention are limited to methods in which the keratin fibers have not been treated with a keratin-reducing compound within a period of up to 7 days prior to the application of compositions (C) and (S) used according to the invention.

Preferably, the keratin fibers are dried after rinsing out compositions (C) and (S). Drying can take place without the active supply of heat. However, drying can also be carried out with a supply of heat at a temperature of 25-120 °C, particularly preferably at a temperature of 30-80 °C, extremely preferably at a temperature of 35-60 °C. Heat is preferably supplied by a heat lamp, a drying rod, a drying hood, a straightening iron, or a hairdryer.

The keratin fibers can be dried after each rinsing step using an absorbent cloth, such as a towel. The towel-dried hair can optionally also be dried partially or completely with a hairdryer or other heat source. It is also possible to let the keratin fibers air dry.

A further feature of the dyeing method according to the invention is that, after application to the keratin fibers, composition (C) is left to act on them for a period of 1 to 60 minutes, preferably 5 to 45 minutes, particularly preferably 20 to 35 minutes, extremely preferably 25 to 30 minutes.

After the development time for composition (C) has elapsed, the keratin fibers are rinsed with water to wash out composition (C).

A further feature of the dyeing method according to the invention is that, after application to the keratin fibers, composition (S) is left to act on them for a period of 0.5 to 60 minutes, preferably 5 to 45 minutes, particularly preferably 20 to 35 minutes, extremely preferably 25 to 30 minutes.

After the development time for composition (S) has elapsed, the keratin fibers are rinsed with water to wash out composition (S).

Further dyeing methods that are preferred according to the invention are characterized in that no oxidation dye precursors are used therein. Typical oxidation dye precursors are p-aminophenol, 4-amino-3-methylphenol, 4-amino-2-aminomethylphenol, 4-amino-2-(1,2-dihydroxyethyl) phenol, 4-amino-2-(diethylaminomethyl) phenol, 2-(2,5-diaminophenyl) ethanol, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl) propyl]amine, N,N′-bis-(2-hydroxyethyl)-N, N′-bis-(4-aminophenyl)-1,3-diamino-propan-2-ol, bis-(2-hydroxy-5-aminophenyl) methane, 1,3-bis-(2,5-diaminophenoxy) propan-2-ol, N,N′-bis-(4-aminophenyl)-1,4-diazacycloheptane, 1,10-bis-(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo-[1,2-a]-pyrazol-1-one, 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy) ethanol, 1,3-bis(2,4-diaminophenoxy) propane, 1-methoxy-2-amino-4-(2-hydroxyethylamino)benzene, 1,3-bis(2,4-diaminophenyl) propane, 2,6-bis(2′-hydroxyethylamino)-1-methylbenzene, 2-({3-[(2-hydroxyethyl)amino]-4-methoxy-5-methylphenyl}amino) ethanol, 2-({3-[(2-hydroxyethyl)amino]-2-methoxy-5-methylphenyl}amino) ethanol, 2-({3-[(2-hydroxyethyl)amino]-4,5-dimethylphenyl}amino) ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline and 7-hydroxyindoline.

In order to also make compositions (C) according to the invention have an attractive scent for the user, further compositions (C) and (S) that are particularly preferably used according to the invention are characterized in that they each contain, independently of one another, at least one perfume oil containing at least one fragrance compound or odorant compound. The definition of an odorant in the context of the present application corresponds to the definition conventional to one skilled in the art, as can be gathered from ROMPP Chemie Lexikon [ROMPP's Chemistry Lexicon], as of December 2007. An odorant, then, is a chemical compound having odor and/or taste, which excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule. To differentiate volatile, low-molecular-weight substances which are not regarded and used as an odorant, either conventionally and also in the context of the present application, but primarily as a solvent, such as, e.g., ethanol, propanol, isopropanol, and acetone, from odorants according to the invention, odorants according to the invention have a molar mass of 74 to 300 g/mol, contain at least one osmophoric group in the molecule, and have an odor and/or taste; in other words, they stimulate the receptors of the hair cells of the olfactory system. Examples of fragrance and odorant compounds of the ester type are benzyl acetate, phenoxyethyl isobutyrate, p-tert-butylcyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, Floramat, Melusat, and Jasmecyclat.

Examples of fragrance and odorant compounds of the ether type are benzyl ethyl ether and Ambroxan; examples of fragrance and odorant compounds of the aldehyde type are the linear alkanals having 8-18 C atoms, citral, citronellal, citronellyl oxyacetaldehyde, cyclamen aldehyde, lilial, and bourgeonal; examples of fragrance and odorant compounds of the ketone type are the ionones, alpha-isomethyl ionone, and methyl cedryl ketone; examples of fragrance and odorant compounds of the alcohol type are anethol, citronellol, eugenol, geraniol, linalool, phenyl ethyl alcohol, and terpineol; examples of fragrance and odorant compounds of the terpene type are limonene and pinene. Examples of fragrance and odorant compounds are pine oil, citrus oil, jasmine oil, patchouli oil, rose oil, ylang-ylang oil, clary sage oil, chamomile oil, clove oil, mint oil, cinnamon leaf oil, lime blossom oil, juniper berry oil, vetiver oil, olibanum oil, galbanum oil, labdanum oil, orange blossom oil, neroli oil, orange-peel oil, and sandalwood oil, further essential oils such as angelica root oil, anise oil, arnica blossom oil, basil oil, bay oil, bergamot oil, champaca flower oil, silver fir oil, silver fir cone oil, elemi oil, eucalyptus oil, fennel oil, fir needle oil, geranium oil, gingergrass oil, guaiacum oil, gurjun balsam oil, helichrysum oil, ho oil, ginger oil, iris oil, cajeput oil, calamus oil, chamomile oil, camphor oil, canaga oil, cardamom oil, cassia oil, pine-needle oil, copaiva balsam oil, coriander oil, curled mint oil, caraway oil, cumin oil, lavender oil, lemongrass oil, lime oil, mandarin oil, melissa oil, musk seed oil, myrrh oil, clove oil, niaouli oil, orange oil, oregano oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, juniper berry oil, wormwood oil, wintergreen oil, hyssop oil, cinnamon oil, citronella oil, lemon oil, and cypress oil. Other fragrance and odorant compounds are ambrettolide, alpha-amyl cinnamaldehyde, anethol, anisaldehyde, anisyl alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methyl anthranilic acid methyl ester, p-methyl acetophenone, methyl chavicol, p-methyl quinoline, methyl β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl-aldehyde, nonyl alcohol, n-octyl aldehyde, p-oxyacetophenone, pentadecanolide, β-phenyl ethyl alcohol, phenyl acetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratrum aldehyde, cinnamaldehyde, cinnamyl alcohol, cinnamic acid, cinnamic acid ethyl ester, and cinnamic acid benzyl ester.

Other (more volatile) odorants are alkyl isothiocyanates (alkyl mustard oils), butanedione, limonene, linalool, linayl acetate and linayl propionate, menthol, menthone, methyl-n-heptenone, phellandrene, phenylacetaldehyde, terpinyl acetate, citral, and citronellal.

Preferably, mixtures of different fragrances are used, which together produce an attractive fragrance note.

Suitable perfume oils can also contain natural odorant mixtures, as are obtainable from plant or animal sources, e.g. pine, citrus, jasmine, rose, lily or ylang-ylang oil. Essential oils having lower volatility, which are usually used as aroma components, are also suitable as perfume oils, such as sage oil, chamomile oil, melissa oil, mint oil, cinnamon leaf oil, lime blossom oil, juniper berry oil, vetiver oil, olibanum oil, galbanum oil, laudanum oil, clove oil, isoeugenol, thyme oil, bergamot oil, geranium oil and rose oil.

Compositions (C) and (S) that are extremely preferably used according to the invention are characterized in that they each contain, independently of one another, at least one fragrance in a total amount of 0.01-5 wt. %, preferably 0.1-3 wt. %, particularly preferably 0.5-2 wt. %, extremely preferably 1-1.5 wt. %, each based on the weight of composition (C) and (S).

The expression “each independently of one another” also includes the case where only one of the two compositions (C) and (S) used according to the invention contains the corresponding optional ingredient.

The present invention also relates to a kit for non-oxidatively dyeing keratin fibers, in particular human hair, comprising a composition C containing powdered leaves of Camellia sinensis in a cosmetic carrier and a composition S containing an aqueous solution of a silver salt.

The statements made regarding preferred embodiments of the dyeing method according to the invention apply, mutatis mutandis, to preferred embodiments of the kit according to the invention and its components, composition C and composition S.

### EXEMPLARY EMBODIMENTS

The exemplary embodiments presented below are intended to explain the subject matter of the invention in more detail, without limiting it thereto.

The dyeing method according to the invention was carried out on white buffalo belly hair strands (tied together in a round bundle, approx. 8 cm of free hair).

A 1 wt. % aqueous solution of matcha (Camellia sinensis) in water adjusted to a pH of 3±0.15 with 10 wt. % hydrochloric acid (measured at 20 °C) was produced as composition C used according to the invention.

Buffalo belly hair strands were immersed in this solution for 30 minutes while stirring. The liquor ratio (amount of matcha solution per gram of hair) was 50 ml matcha solution per gram of hair.

Once treatment with Camellia sinensis had finished, the hair strands were rinsed under running deionized water for 30 seconds while being combed 20 times (20 °C).

The hair strands were then dried with a commercially available hair dryer at a defined distance (d=10 cm) and a defined temperature (T=80±5 °C) while combing them 20 times.

Once treatment with Camellia sinensis had finished, a change in the color of the hair fibers was not detectable.

In the following step of the dyeing method according to the invention, the strands were immersed for a further 30 minutes in an aqueous solution of 0.5 wt. % silver nitrate (AgNO₃) which had a pH of 4.5 measured at 20 °C. The liquor ratio (amount of silver nitrate solution per gram of hair) was 50 ml of silver nitrate solution per gram of hair. After the development time had elapsed, the strands were washed again, as described above. The strands were then dried so that a residual moisture content of approximately 23 wt. % water remained in the hair fibers based on their weight.

The freshly dyed strands showed a very slight red shimmer when sunlight was eliminated during the dyeing process.

In the last step of the dyeing method preferred according to the invention, the strands were exposed to sunlight. Sunlight that reaches the earth has radiation in the wavelength range of approximately 290 nm to 700 nm. After approximately 10 minutes to 90 minutes of sunlight exposure (behind window glass, July, in Hamburg), an intense red was achieved as the resulting color. The color developed from an orange and light-red tone to a strong, dark-red tone. The same dyeing results were obtained under the following standardized conditions in a color assessment cabinet: two hours exposure time in a color assessment cabinet. The color assessment cabinet contained fluorescent lamps (L 58 W/954, LUMILUX DE LUXE T8). These had the following properties: luminous efficacy 78 lm/W (lumens per watt); luminous flux 4550 lm; color temperature 5400 K, and served a wavelength spectrum of approximately 300 nm-760 nm.

The preparation of the treatment compositions C and S and the treatments of the hair strands with these compositions C and S were carried out at room temperature (20-25 °C).

### Determination of the Shade Achieved by the Method According to the Invention

All colorimetric measurements were carried out using the Spectraflash SF 600 colorimetric device from Datacolor.

The color difference, also known as dE or ΔE, can be easily determined colorimetrically using a colorimeter that measures colors in the L*, a, b* color space—for example, a Datacolor colorimeter, type Spectraflash SF 600.

The L*, a*, b* color space refers to the CIELAB color space. The L value represents the brightness of the color (black-white axis); the greater the L value, the brighter the color. The a value represents the red-green axis of the system; the greater this value, the more the color is shifted towards red. The b value represents the yellow-blue axis of the system; the greater this value, the more the color is shifted towards yellow.

The color shift ΔE, i.e., the color difference between two (hair) colors, for each of which an L*, a*, b* value combination was determined, is calculated according to the following formula:

ΔE=(ΔL²+Δa²+Δb²)^{0.5}

The greater the value for ΔE, the more pronounced the color difference.

For the spectrophotometer measurements, a D65 lamp and a diffuse/8° optical configuration were used. The spectral reflection data for each sample from 380 nm to 700 nm were converted to colorimetric data using DCI Color software. Reflection measurements were taken for each hair sample, with the average of 4 measurements being recorded.

The color difference (ΔE) between undyed strand and dyed strand was calculated according to the following formula:

ΔE=\sqrt{(Lv−Ln)²+(av−an)²+(bv−bn)²}, where
Lv, av, bv: Colorimetry values for dyed strands (first Camellia sinensis, then silver nitrate)
Ln, an, bn: Colorimetry values for undyed, untreated strands (before Camellia sinensis)

TABLE 1
L*, a*, b* values of the untreated hair strands

| | | | |
|---|---|---|---|
| Batch Name | CIE L sample | CIE a sample | CIE b sample |

Day 1

| | | | |
|---|---|---|---|
| Untreated 1 | 79.6 | −0.83 | 8.64 |
| Untreated 2 | 79.79 | −1.25 | 7.81 |
| Untreated 3 | 79.43 | −0.95 | 8.11 |
| Average | 79.61 | −1.01 | 8.19 |

TABLE 2
L*, a, b*, ΔE values of the Camellia sinensis/silver
nitrate dyes followed by sunlight exposure

| | CIE L | CIE a | CIE b | |
|---|---|---|---|---|
| Batch Name | sample | sample | sample | ΔE |
| Day 1 | | | | sample |
| Matcha + Ag_1 | 22.65 | 12.76 | 9.35 | 58.55 |
| Matcha + Ag_2 | 24.11 | 12.37 | 8.31 | 57.32 |
| Matcha + Ag_3 | 24.11 | 15.91 | 9.65 | 57.85 |
| Average | 23.62 | 13.68 | 9.10 | 57.89 |

1. A method for non-oxidative dyeing of keratin fibers, the method comprising:
providing a composition comprising powdered leaves of Camellia sinensis in a cosmetic carrier;
applying the composition comprising powdered leaves of Camellia sinensis to the keratin fibers;
maintaining the composition comprising powdered leaves of Camellia sinensis in the keratin fibers for a time of from 1 to 60 minutes;
rinsing the keratin fibers for a first time;
applying a composition comprising an aqueous solution of a silver salt to the keratin fibers for a time of from 0.5 to 60 minutes; and
rinsing the keratin fibers for a second time,
wherein no oxidizing agents other than atmospheric oxygen are used in the method.

2. The method of claim 1, further comprising exposing the keratin fibers to electromagnetic radiation after rinsing the keratin fibers for the second time.

3. The method of claim 1, wherein the keratin fibers, after rinsing the keratin fibers for the second time, have a water content of from 1-42 wt. %, based on the total weight of the keratin fibers.

4. The method of claim 2, wherein the electromagnetic radiation is electromagnetic radiation from a natural light source.

5. The method of claim 2, wherein the keratin fibers are exposed to the electromagnetic radiation for a time of from 0.5-60 minutes.

6. The method of claim 1, wherein the powdered leaves of Camellia sinensis are in an amount of from 0.2-50 wt. %, based on the total weight of the composition comprising powdered leaves of Camellia sinensis.

7. The method of claim 1, wherein the composition comprising powdered leaves of Camellia sinensis has a pH of 2.0 to 8.0, measured at 20 °C.

8. The method of claim 1, wherein the composition comprising powdered leaves of Camellia sinensis comprises water in an amount of from 30.0-99.8 wt. %, based on the total weight of the composition comprising powdered leaves of Camellia sinensis.

9. The method of claim 1, wherein the silver salt is selected from the group consisting of silver nitrate, silver sulfate, silver citrate, silver dihydrogen citrate, silver lactate, silver acetate, silver malate, silver succinate, silver tartrate, silver mandelate, silver salicylate, silver gluconate, silver adipate, silver galactarate, and any mixture thereof.

10. The method of claim 1, wherein the silver salt is in an amount of from 0.05-2 wt. %, based on the total weight of the aqueous solution of the silver salt.

11. The method of claim 1, wherein the aqueous solution of the silver salt has a pH of from 2.0 to 8.0 measured at 20 °C.

12. A kit for non-oxidative dyeing of keratin fibers, the kit comprising:
a composition comprising powdered leaves of Camellia sinensis in a cosmetic carrier; and
a composition comprising an aqueous solution of a silver salt.

13-14. (canceled)

15. The method of claim 1, wherein the keratin fibers are human hair.

16. The method of claim 1, further comprising drying the keratin fibers after rinsing the keratin fibers for the first time.

17. The method of claim 1, further comprising drying the keratin fibers after rinsing the keratin fibers for the second time.

18. The method of claim 6, wherein the powdered leaves of Camellia sinensis are in an amount of from 1-2 wt. %, based on the total weight of the composition comprising powdered leaves of Camellia sinensis.

19. The method of claim 9, wherein the silver salt is a mixture of the silver nitrate, the silver sulfate, the silver citrate, the silver dihydrogen citrate, and the silver lactate.

20. The method of claim 10, wherein the silver salt is in an amount of from 0.3-0.5 wt. %, based on the total weight of the aqueous solution of the silver salt.

21. The kit of claim 12, wherein the keratin fibers are human hair.

22. The kit of claim 12, wherein the powdered leaves of Camellia sinensis are in an amount of from 1-2 wt. %, based on the total weight of the composition comprising powdered leaves of Camellia sinensis.
